(19) United States
(12) Patent Application Publication
(10) Pub. No.: US 2022/0347000 A1
(43) Pub. Date: Nov. 3, 2022

(54) LOAD DISTRIBUTION DEVICE FOR PREVENTING ORTHOSIS MIGRATION

(71) Applicant: Icarus Medical, LLC, Charlottesville, VA (US)

(72) Inventors: David T. Johnson, Charlottesville, VA (US); Philip Miller, Charlottesville, VA (US); Carter Kitchin, Charlottesville, VA (US)

(73) Assignee: Icarus Medical, LLC, Charlottesville, VA (US)

(21) Appl. No.: 17/864,675

(22) Filed: Jul. 14, 2022

Related U.S. Application Data
(63) Continuation-in-part of application No. 17/700,479, filed on Mar. 21, 2022.
Application No. 17/700,479 is a continuation-in-part of application No. 17/537,476, filed on Nov. 29, 2021.
Application No. 17/537,476 is a continuation-in-part of application No. 17/211,635, filed on Mar. 24, 2021.
Application No. 17/211,635 is a continuation of application No. 17/074,542, filed on Oct. 19, 2020.
Application No. 17/074,542 is a division of application No. 15/585,968, filed on May 3, 2017, now Pat. No. 10,806,619.
Application No. 17/537,476 is a continuation-in-part of application No. 17/074,571, filed on Oct. 19, 2020.
Application No. 17/074,571 is a continuation-in-part of application No. 15/585,968, filed on May 3, 2017, now Pat. No. 10,806,619.
(60) Provisional application No. 62/331,315, filed on May 3, 2016.
(60) Provisional application No. 62/331,315, filed on May 3, 2016.

(51) Int. Cl. A61F 5/01

(52) U.S. Cl. CPC A61F 5/0125; A61F 2005/0139; A61F 2005/0165

(57) ABSTRACT

A device to prevent migration of an orthotic, such as an unloading joint brace.

### CROSS-REFERENCE TO RELATED APPLICATIONS

The present application is a continuation-in-part of and relies on the disclosures of and claims priority to and the benefit of the filing date of U.S. patent application Ser. No. 17/700,479 filed Mar. 21, 2022, which is a continuation-in-part of and relies on the disclosures of and claims priority to and the benefit of the filing dates of U.S. patent application Ser. No. 17/537,476 filed Nov. 29, 2021, U.S. patent application Ser. Nos. 17/074,571 and 17/074,542 filed Oct. 19, 2020, U.S. patent application Ser. No. 17/211,635 filed Mar. 24, 2021, which rely on the disclosures of and claim priority to and the benefit of the filing date of U.S. patent application Ser. No. 15/585,968, filed May 3, 2017, which claims priority to and benefit from U.S. Provisional Patent Application No. 62/331,315 filed on May 3, 2016. The disclosures of those applications are hereby incorporated by reference herein in their entirety.

### BACKGROUND OF THE INVENTION

### Field of the Invention

The current invention provides a load distribution device that supplements orthotic attachment systems. The load distribution device is particularly effective at distributing and redirecting the detrimental forces associated with, for example, dynamic orthoses, thereby minimizing migration, increasing wearer comfort, and improving the efficacy of the orthosis.

### Description of Related Art

Solutions for orthosis migration have been proposed with varying success. Most involve enhancing the friction between the straps or attachment system and the wearer's leg either by increasing the contact area, increasing the tension of the strap, increasing the coefficient of friction between the strap or connector and the wearer, or combinations thereof.

For example, anti-migration straps for a knee brace are described in the art. The straps described are flexible and may be made or coated with a high-friction material. In some versions it is recommended that the anti-migration strap is disposed over the widest portion of the wearer's calf. Such an anti-migration device does not redirect the forces that lead to migration but instead uses friction to withstand them, which is therefore unlike the current invention.

Another example known in the art is an anti-migration wrap that is secured to an orthotic strap made from a flexible material, such as a breathable foam with high friction properties. The wrap may be made from a unitary material or combinations of materials such as a core of foam, and skinned with a high friction material. Like the previous example, this solution uses friction to withstand the anti-migration forces, but does not redirect the forces; again, it is therefore unlike the current invention.

Similarly, it is known to use under-wraps or sleeves to help secure strapping or attachment systems to limbs. The sleeves fit tightly to the limb and offer a large surface area to help withstand migration forces. By selecting the right material for the outer surface of the sleeve they can help prevent the migration of the orthotic straps. Sleeves, however, may be uncomfortable, difficult to don or doff, and are not appropriate to be worn over clothing.

Examples of specialized solutions such as buttressing or protrusions also exist but these are specific to braces or body geometries (e.g., a buttress for an ankle foot orthotic) and are not general solutions to the problem of strap migration.

Anti-migration and redirecting attachment system forces are important for all types of orthoses but are even more difficult to achieve simultaneously for dynamic orthoses. The forces required for therapeutic manipulation of the body part can be quite high. A brace as described herein can produce 40 lbs. of unloading force; a heavy-duty brace as described herein can produce an even greater unloading force. However, the same beneficial forces that are needed for therapeutic manipulation also have the undesired effect of moving/migrating the orthotic out of proper position on the wearer and making it uncomfortable to wear.

Therefore, an improved load distribution device with attachment system is needed that can effectively distribute therapeutic manipulation forces without compromising security, convenience, and comfort of the underlying orthotic and its attachment system.

Examples of references related to this art include the following U.S. patents and patent applications: U.S. Pat. Nos. 9,265,642; 10,285,842; 10,702,409; 10,786,381; 10,806,620; 11,096,816; 7,850,632; 11,253,384; U.S. Publication No. 20200000620A; U.S. Pat. No. 7,918,812; U.S. Publication No. 20120065562A; and U.S. Pat. No. 5,554,104.

### SUMMARY OF THE INVENTION

In general, orthotics can be divided into three categories: immobilizing, stabilizing, and dynamic. An immobilizing orthotic, such as a sling or a splint, holds the limb or body part in a fixed position. (The phrases body part, limb, and body element are used interchangeably herein.) The purpose of an immobilizing orthotic is to prevent injury and/or aid in healing by keeping the muscles, joints, and bones in a specific position-either to align various components (e.g., the ends of a fractured bone), or to prevent motion of damaged components (e.g., a severe muscle strain).

Stabilizing orthotics allow some motion of the affected joint or limb, but they protect the joint from unwanted range of motion. For example, a knee orthotic can be used to provide lateral stability of the knee while allowing full freedom of flexion/extension motion. Another example of a stabilizing orthotic is an elbow brace used to prevent hyper extension of the elbow while allowing normal range of motion of the joint.

The third type of orthotic is a dynamic orthotic. These orthoses are used to manipulate a limb or body part therapeutically by applying specific force vectors. Dynamic orthoses can be used to reduce pain or improve function. For example, an unloading knee brace, such as the Ascender™ sold by Icarus Medical Innovations of Charlottesville, Va., imparts forces to the wearer's thigh and calf that reduce the applied forces on the wearer's patella femoral joint as described in U.S. Pat. No. 10,806,619, and incorporated herein by reference in its entirety. Immobilizing and stabilizing braces dominate the bracing market, with dynamic bracing representing only a small fraction, in part because it is a newly developed technology. In a dynamic brace, much higher forces are generated over a small surface area. Available strapping or attachment technology used for immobilizing and stabilizing braces are currently unsuitable or have not yet been developed, and therefore, not readily adaptable to dynamic orthosis that require higher load distribution, and possible direction of force. The problems are in whole or in part resolved or improved upon by the invention described herein.

All three types of orthotics need to be secured to the wearer's body to work as intended. Orthosis attachment systems include, but are not limited to, straps, webbing, pads, belts, laces, cords, sleeves, cuffs, slings, elastics, buckles, stops, clips, rings, and the like, which are common methods of/mechanisms for securing orthoses/orthotic braces. Therefore, the various kinds and combinations of orthosis attachment mechanisms/means such as straps, webbing, buckles, stops, etc. used to secure an orthotic to the wearer, as noted above and herein, will be henceforth referred to in aggregate as attachment system, strapping system, or orthosis attachment system. Consequently, the term orthosis attachment system(s) and attachment system(s) include but are not limited to mechanisms for attaching an orthosis to a wearer, such as straps, webbing, pads, belts, laces, cords, sleeves, cuffs, slings, elastics, buckles, stops, clips, rings, straps, or combinations thereof.

Orthosis attachment systems should be secure (for example, resist sliding out of place on the limb to which they are affixed), convenient (for example, easy to don and doff), and comfortable (for example, not cause chafing). These three attributes are somewhat contradictory. Tightening an orthosis attachment system sufficiently to prevent the orthotic from sliding may make it uncomfortable to wear for extended periods of time. Devices for increasing and/or decreasing tension may also be used in conjunction with the load distribution device, such as a rotary tensioning dial, where the dial may be mounted on the load distribution device or on the orthosis to which it is attached. Elastic tensioning bands may also be used in-line with this system, or as part of the attachment system. The load distribution device may be attached in a hinging manner that clasps around a body part, such as mating body part. It may also lock in place with a ratchet-pawl system or a similar locking mechanism that allows securing the device to the orthosis, and/or for tightening the orthosis to the body part.

The unwanted displacement of an orthosis on a limb or body part is called migration and is a common problem. It occurs when the orthosis (or parts of the orthosis attachment system) slip either on the wearer's skin or the clothing they are wearing under the orthosis. The anti-migration solution described herein is effective for both strap-to-skin and strap-to-clothing interfaces.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1 is a depiction of a human limb and force vector illustration.

FIG. 2 is a depiction of an orthotic knee brace.

FIG. 3 is a depiction of an orthotic knee brace and force vector illustration, and FIG. 3a is a depiction of a load distribution device positioned on a wearer's calf, according to an embodiment of the current invention.

FIG. 4 is a depiction of a load distribution device positioned on a wearer's calf, according to an embodiment of the current invention.

FIGS. 5 and 5a are depictions of an existing strap.

FIGS. 6 and 6a are depictions of a load distribution device according to an embodiment of the current invention.

FIG. 7 is a depiction of a load distribution device according to an embodiment of the current invention.

FIG. 8 is a depiction of a load distribution device according to an embodiment of the current invention.

FIG. 9 is a depiction of a load distribution device according to an embodiment of the current invention.

### DETAILED DESCRIPTION OF THE INVENTION

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

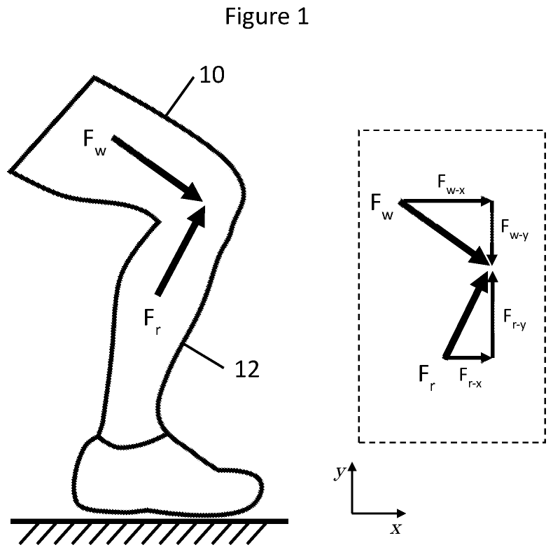

FIG. 1 shows the side view of a knee joint in a partially flexed position. For a person suffering from patellofemoral osteoarthritis (“PFOA”), the cartilage in the patella femoral compartment is damaged or missing which allows bone-on-bone contact. This is especially a painful when weight is applied while the knee is being flexed (for example, going up or down stairs). The force vector, Fw, from the person's weight is supported by the thigh (10). There is a resulting force vector from the floor, Fr, that is supported by the lower leg (12). The two forces can be resolved into the x and y coordinates as shown in dashed box. Thus, there are two forces, Fw-y and Fr-y, that press the knee bones together which causes pain.

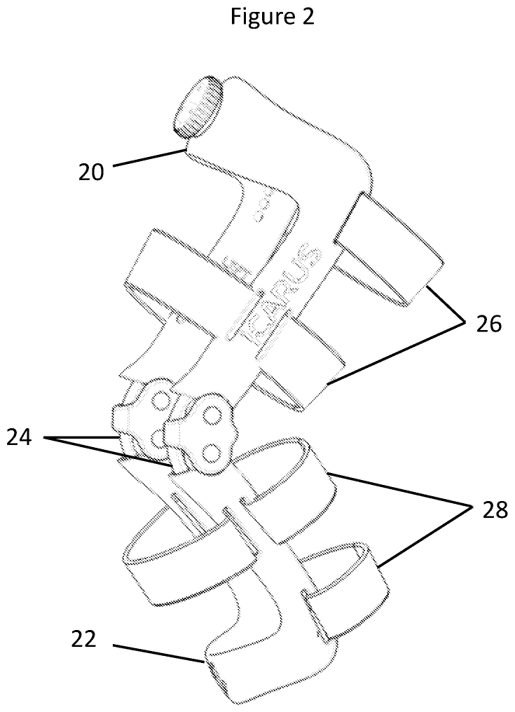

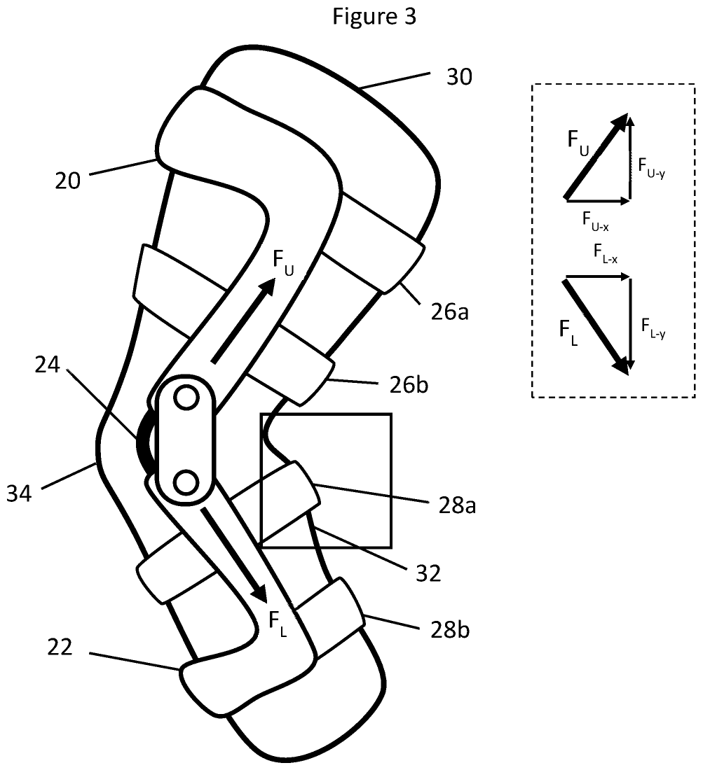

The unloading knee brace shown in FIG. 2 has two energy storage elements (24) that are connected to the upper frame assembly (20) and the lower frame assembly (22). The upper strapping elements (26) and lower strapping elements (28) comprise, in embodiments, the orthosis attachment system of the brace/orthosis. When the brace is flexed, the energy storage elements are engaged and provide a counterforce that unloads the forces on the knee. The brace, in aspects, has been shown to be effective at reducing PFOA pain. In a study of 30 wearers of the illustrated brace who suffered from PFOA, the reduction in pain while wearing the pictured brace on the KOOS scale was statistically the same as the reduction in pain reported from a knee replacement.

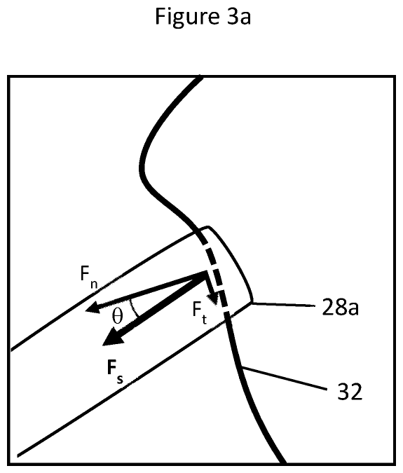

The force vectors from the energy storage element on the brace pictured in FIG. 2 are shown schematically in FIG. 3. When the leg (30) is bent at the knee (34), the energy storage element (24) is elongated and applies a tensile force on the upper and lower brace elements (20 and 22, respectively). The forces, Fu and Fl, on the upper and lower braces oppose the tensile forces generated by the energy storage elements. The orthosis attachment system comprising straps (26a) and (26b) and the lower leg straps (28a) and (28b) is designed to resist these forces and hold the brace securely in place. By this means, the brace transfers the unloading force to the wearer's thigh and lower leg thereby unloading the knee joint.

FIG. 3a is an enlargement of the box drawn in FIG. 3. The lower leg strap (28a) wraps around the wearer's calf (32). The outline of the wearer's leg is shown in FIG. 3a as a dotted line where it is hidden by the strap. The vector Fs represents the tensile force of the strap on the leg. Fs can be resolved into the vector Fn which is the force normal to the surface of the wearer's leg and Ft which is the force tangential to the surface of the wearer's leg at the location of the strap (28a). Ft is the force responsible for the migration of the strap/orthosis.

In FIG. 3a, the offset of the direction of Fs from the normal vector of the surface of the leg is shown as the angle θ. When 0 is small then the migration force, Ft, is negligible. In aspects, migration can be a problem when 0 is large due to the strap contacting a body part of high curvature (for example, a calf, shoulder, or ankle)—or when there is misalignment between direction of the straps' tensile forces and the desired therapeutic manipulation forces.

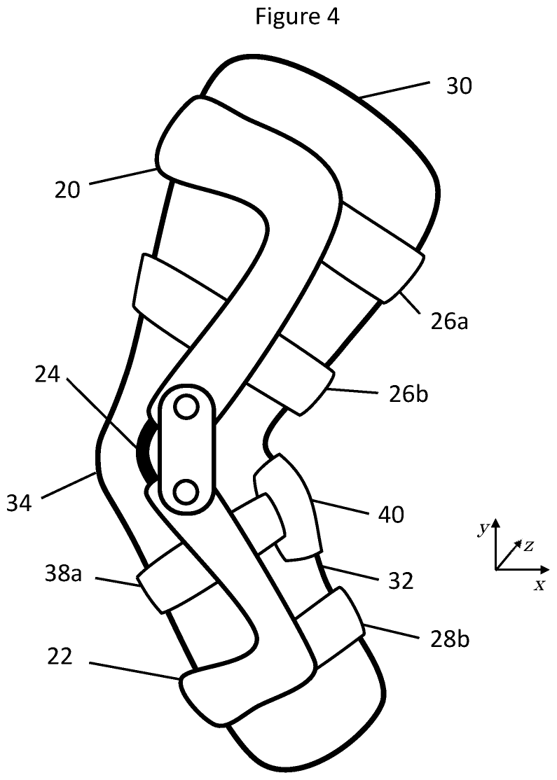

FIG. 4 shows a schematic of a load distribution device, 40, positioned on a wearer's calf, 32. The load distribution device is particularly adept at overcoming migration forces because it curves and conforms to a surface of the calf along two orthogonal planes—around the width of the calf in the x-z plane and along the length of the calf in the x-y plane. In embodiments, this can be accomplished by taking a three-dimensional (“3D”) scan of the wearer's limb, deriving the dimensions of the limb from the scan, and altering the geometry of the load distribution device to substantially approximate the surface contour of the limb where the load distribution device contacts the limb thereby ensuring that the load distribution device conforms/cups/wraps/supports the limb to substantially approximate/match/conform to the limb or mating body part (e.g., medically, clinically, nearly, mostly, or precisely approximate/match/conform to the limb or mating body part). The resulting geometry is then fabricated using one of the suitable manufacturing techniques known in the art, such as 3D printing or additive manufacturing.

The invention shown in FIG. 4 can be superior to straps or pads because it conforms to the wearer's limb in two directions whereas traditional straps and pads are only flexible in one direction.

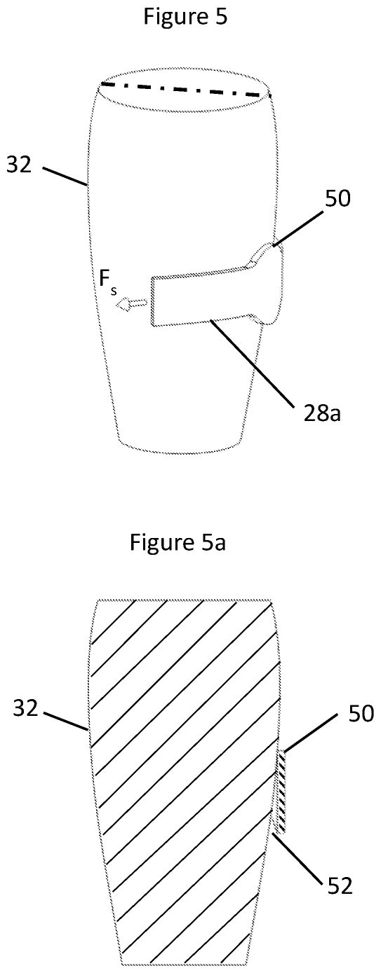

FIG. 5 is a schematic of a strap (28a) with an enlarged contact patch (50) typical of the art. The strap and contact patch wrap around the calf of a leg (32) and are pulled in tension as shown by the force vector, Fs. A contact patch can be fabricated by either widening of the strap at a specific location or by adding a pad, bolster, swatch, or the like to the strap. Traditional contact patches are made from flexible materials such as leather, foam, rubber, fabric, non-wovens, webbing, etc. These materials conform to the limb primarily in the direction of the long axis of the strap. They serve to increase the contact area between the strap and the limb, increasing the friction which hinders the strap from migrating and also spreads the strapping forces for increased comfort.

FIG. 5a is a cross-sectional view taken along the dashed line shown in FIG. 5. Although the strap and contact patch conform to the leg along the long axis of the strap as shown in FIG. 5, there is a gap (52) at the lower edge of the strap between the contact patch and the leg. For a body part like a thigh or upper arm, the underlying muscle and skin can deform somewhat to close the gap and help make better contact. For body parts with little underlying musculature (such as elbows, shoulders, or ankles) or for the large forces associated with dynamic orthoses, the solutions already known in the art are not sufficient to prevent migration.

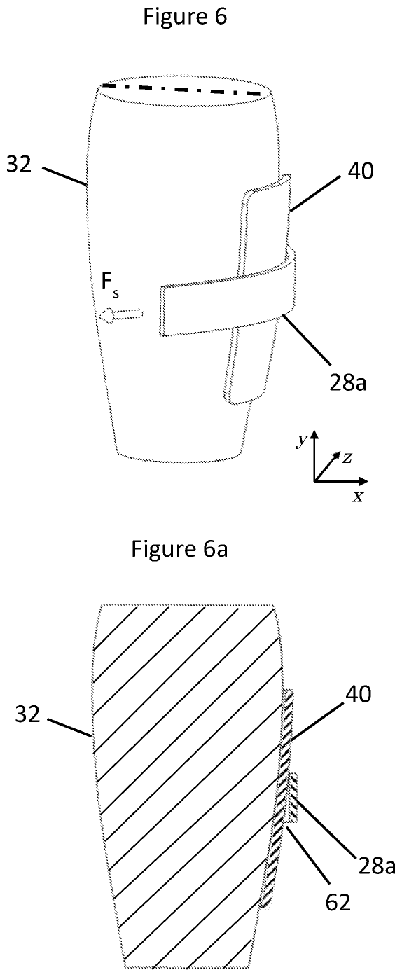

FIG. 6 is a schematic of an improved load distribution device according to the current invention. The load distribution device curves around the limb in two orthogonal planes. It cups the limb in the x-z plane and cradles it in the x-y plane. The cross section shown in FIG. 6a demonstrates how the at least two-fold curvature is superior to conforming to the limb/mating body part compared to a traditional contact patch.

In aspects, the load distribution device is made of a rigid or semi-rigid material in order to transfer and redirect the unloading forces effectively. Using a rigid or semi-rigid material has the added benefit that it is possible to accommodate incomplete contact between the strap and the load distribution device. The gap (62) at the lower edge of the strap in FIG. 6a between the strap (28a) and the load distribution element (40) is similar to the gap in FIG. 5a between the strap (28a) and the leg (32). In embodiments, because the load distribution device according to the present invention is rigid or semi-rigid it is able to transmit the tensile force of the strap, Fs, without creating a pressure point at the top the strap, such as a detrimental pressure point. In embodiments, the strap can be constrained on the load distribution device (via an attachment/connection mechanism or a shape of the load distribution device, such as the device having slots, apertures, holes, slits, tabs, adhesives, hooks, fasteners, etc.) to prevent migration between the orthosis attachment system and the load distribution device.

A shape of the load distribution device can be dependent on several factors, such as the magnitude and direction of migration forces that need to be overcome, the necessary movement of the underlying muscles and skin it is contacting (for example, the normal flex of the calf muscle when walking), the range of tightness of the adjoining strap, the material(s) used to fabricate the load distribution device, and so on. However, in general, the contact area, A, should be

A>=Ft/(P×m)

where Ft is the migration force, P is the pressure applied by the load distribution device due to the strap tension, and m is the coefficient of friction at the load distribution device/limb interface. As the migration forces increase, the contact area of the load distribution device needs to increase to compensate.

It may be advantageous, in aspects, to cover the surfaces of the load distribution device with one or more of a variety of materials. For example, a thin conformable foam pad can provide additional cushioning. A chamois covering can absorb sweat and prevent chafing. Gilly cloth can provide camouflage. Coverings and other modifications can be selected such that they do not inhibit the ability of the load distribution device to prevent migration. Silicone can be used.

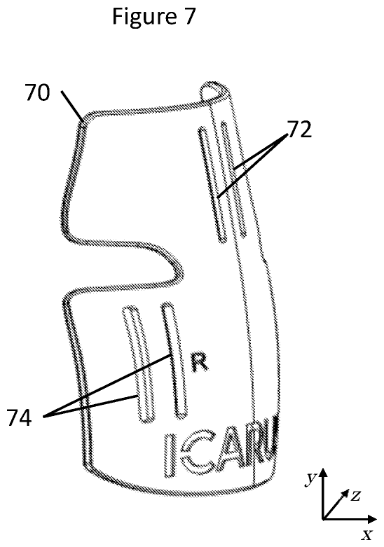

FIG. 7 is an orthogonal view of an example of a load distribution device (70) that in testing has been shown to resist the migration forces due to the therapeutic manipulation forces of a knee brace. By way of example only, the contact area of the inner surface of the load distribution device is around 125 cm². The radius of curvature in the x-y plane is around 40 cm. The radius of curvature in the x-z plane is around 6 cm. The shape of the element is roughly trapezoidal where the length of the top edge is around 8 cm, the length of the bottom edge is around 12 cm, and the length of the side edges are around 13 cm. Again, these dimensions are non-limiting examples only. The strap (28a) shown in FIGS. 6 and 6a can be constrained or otherwise attached to/connected to the load distribution device by the slots (72) shown in FIG. 7, and strap (28b) shown in, e.g., FIG. 4 can be constrained or otherwise attached to/connected to the load distribution device by slots (74) shown in FIG. 7.

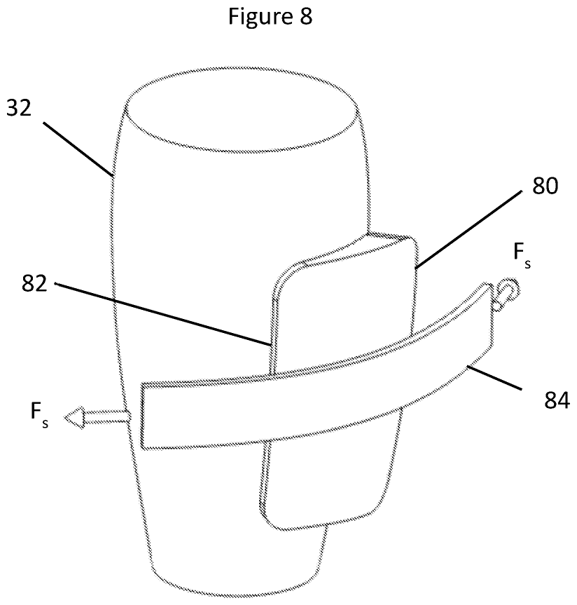

In another embodiment, a load distribution device can be used to contribute to the therapeutic manipulative forces of the orthosis in addition to its anti-migration function. FIG. 8 shows a schematic of a load distribution device according to the present invention that incorporates a wedge element. In aspects, there can be provided a first edge, such as a thicker edge (80), and a second edge, such as a thinner edge (82). The wedge shape can hold the portion of the strap (84) away from the limb/mating body part (32). When tension on the strap is applied as shown by the vectors Fs, a moment can be generated around the thinner edge (82) of the load distribution device. The resulting force can be employed to impart a twisting action to the limb.

The wedge can be placed in other locations on the load distribution device to impart a desired force vector (for example, along the bottom edge to generate a lifting force). A similar effect can be generated by using a mechanical element to lift the strap away from a back surface of the load distribution device, instead of employing a wedge shape.

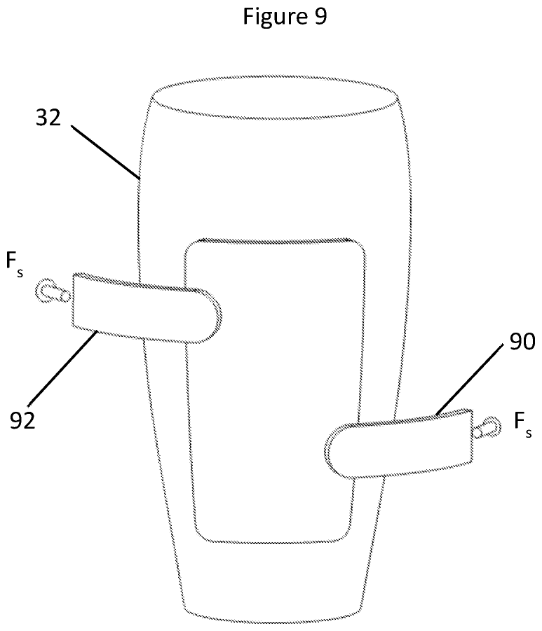

In yet another embodiment, the attachment points of the straps can be offset. As shown in FIG. 9, tension on the straps (90 and 92) would impart a counter-clockwise moment on the load distribution device, which would be transferred to the limb/mating body part (32).

While certain joints, limbs, and body parts have been mentioned herein, it is contemplated that the invention described herein could be applied to the neck, spine, shoulder, arm, elbow, hand, wrist, waist, leg, knee, ankle, foot, and other joints and their associated body parts. Likewise, the invention described herein could be used in conjunction with an orthosis for veterinary purposes and applied to animal joints or parts such as hocks, fetlocks, shanks, tails, etc. which don't have equivalent human parts.

The various embodiments of the load distribution device could be employed separately or in combination to simultaneously prevent the migration of a dynamic orthotic device and deliver the therapeutic manipulation force vectors. The load distribution device can be particularly suitable for the high force vectors needed for dynamic orthoses or where an orthosis strap needs to be positioned on high curvature body parts such as shoulders, calves, forearms, ankles, thighs, and the like.

In embodiments, the presence of a wound or injury on a wearer's limb/body part/joint may interfere with the preferred load distribution device geometry. A conformable sock, wrap, sleeve, elastic cloth, or the like may be employed underneath the load distribution element. The conformable wrap can have a surface area larger than the load distribution device, although that is not always necessary. The conformable wrap could be made as large as needed to provide enough skin-to-wrap (or cloth-to-wrap friction if the wrap is worn over the clothes) to overcome the forces imparted by a dynamic orthotic. The load distribution device, orthotic, and/or both could be coupled with the wrap such that they essentially lock in place. The wrap can provide, in aspects, a high friction to the limb, or friction to the limb. In turn, the orthotic/load distribution device can couple to the wrap so that forces imparted on the orthotic/load distribution device are distributed to the wrap. A rigid or semi-rigid portion of the load distribution device may also be contoured around a wound or sensitive area like an osteophyte or varicose vein, or it can be designed so that a gap or void is used to direct force to other areas of the surface instead of the wound or sensitive area.

For example, if a patient had a wound on the top of their shoulder, a load distribution device for a shoulder orthosis would lie on top of the wound and be uncomfortable to wear. A shoulder load distribution element with a cut-out to avoid direct pressure to the wound may not be able to redirect the orthosis forces adequately. In this case, a neoprene sleeve with a Velcro loop on the outer surface could be worn on the arm up to the shoulder. A shoulder load distribution device could be fashioned with the Velcro hook on the inner side and a cut-out to avoid the wound. By coupling the load distribution device to the sleeve (by the Velcro), the forces could be transmitted or redistributed from the load distribution device to the sleeve, thereby allowing the orthosis to function while still being comfortable to wear. A rotary tensioning dial and elastic tensioning system (or other type of tensioning system with or without elastic components) may direct forces around the shoulder joint using the load distribution device that is conformed to a shoulder, or also to distribute force on other body plates that conform to other body parts. For example, a back brace can utilize one or more load distributors or body plates that may be tensioned to help direct forces across the spine to an improved biomechanical state.

The load distribution device described herein can be adapted to many joints that require force manipulation across the joint, such as hip, elbow, back, ankle, or shoulder. Within the knee, one example is a device that imparts a force on the patella to direct it to a position that is more biomechanically favorable, often medially. In this case a tensioning system that is optionally adjustable may dynamically apply a pushing or pulling force across a knee brace or a sleeve. Concentrated forces directed into or near the patella would be uncomfortable and less effective than a load distribution device that can also direct force.

Another embodiment may use the load distribution device to distract a joint by, for example, pressing the calf away from the quadriceps, where the load distribution device also directs force away from the joint while simultaneously distributing force. For example, the device may tilt in a manner to where the top of the calf “belly” is held tighter to the tibial crest than the lower part of the load distributor, and so the calf then becomes an ideal surface for either preventing brace migration, or generating a counter force that could distract the knee joint.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entirety and as such are intended to provide an efficient way of supplementing the enabling disclosure of this invention as well as provide background detailing the level of ordinary skill in the art.

As used herein, the term “about” or “around” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

What is claimed:

1) A load distribution device for use with an orthosis and an orthosis attachment system, wherein the orthosis attachment system attaches the orthosis to a wearer of the orthosis:
wherein the load distribution device prevents at least one of migration of the orthosis and detrimental localized load force concentrations of the orthosis attachment system;
wherein the load distribution device comprises a rigid or semi-rigid structural support body having a curvilinear surface that substantially approximates or matches a surface of a mating body part in a first direction, wherein the curvilinear surface also substantially approximates or matches the surface of the mating body part in a second direction that is orthogonal to the first direction; and
wherein the load distribution device is connected to the orthosis attachment system or is shaped to attach to the orthosis attachment system.

2) The load distribution device of claim 1, wherein the load distribution device further applies a force in a direction to improve biomechanics of a joint of the wearer of the orthosis.

3) The load distribution device of claim 1, wherein the orthosis is a dynamic orthosis.

4) The load distribution device of claim 1, wherein the curvilinear surface is determined based on a three-dimensional scan of the mating body part.

5) The load distribution device of claim 1, wherein the load distribution device is made using three-dimensional (“3D”) printing or additive manufacturing.

6) The load distribution device of claim 1, wherein the load distribution device is fabricated from at least one of: a rigid or semi-rigid thermoplastic, nylon, polypropylene, polyethylene, acrylonitrile butadiene styrene, polylactic acid, polyethylene terephthalate, polyethylene terephthalate glycol, and polyurethane.

7) The load distribution device of claim 1, wherein the load distribution device is fabricated using a mold of the load distribution device in order to create a cast of the load distribution device made of at least one of carbon fiber, a composite material, and fiberglass material.

8) The load distribution device of claim 1, wherein a surface area of the load distribution device contacting the mating body part is greater than a migration force divided by a coefficient of friction between an inner surface of the load distribution device and the mating body part times a pressure imparted by the load distribution device on the mating body part due to a tension provided by the orthosis attachment system.

9) The load distribution device of claim 1, wherein a surface area of the load distribution device contacting the mating body part is one of: greater than 20 cm², greater than 50 cm², greater than 90 cm², greater than 100 mm², and greater than 120 cm².

10) The load distribution device of claim 1, wherein a surface of the load distribution device contacting the mating body part comprises a high-friction material to minimize migration or translation between the mating body part and the load distribution device.

11) The load distribution device of claim 10, wherein the high-friction material is silicone.

12) A method for preventing migration of an orthosis, preventing detrimental localized load force concentrations of an orthosis attachment system, or both, comprising:
three-dimensionally scanning a surface of a mating body part;
digitally designing a load distribution device that connects to the orthosis attachment system;
fabricating using three-dimensional printing or additive manufacturing the load distribution device, wherein the load distribution device comprises a rigid or semi-rigid structure providing a curvilinear surface that substantially conforms to a surface of the mating body part in a first direction and the surface of the mating body part in a second direction that is orthogonal to the first direction; and
attaching the load distribution device to a wearer using the orthosis attachment system.

13) The load distribution device of claim 12, wherein the load distribution device further applies a force in a direction to improve biomechanics of a joint of the wearer.

14) The method of claim 12, wherein the orthosis is a dynamic orthosis.

15) A load distribution device connected to an orthosis attachment system, the load distribution device comprising:
a rigid or semi-rigid structure providing a curvilinear surface that substantially approximates or conforms to a surface of a mating body part in a first direction and substantially approximates or conforms to the surface of the mating body part in a second direction that is orthogonal to the first direction; and
a moment arm generated by an offsetting wedge located between the orthosis attachment system and the load distribution device.

16) A load distribution device connected to an orthosis attachment system, the load distribution device comprising:
a rigid or semi-rigid structure providing a curvilinear surface that substantially approximates or conforms to a surface of a mating body part in a first direction and that substantially approximates or conforms to the surface of the mating body part in a second direction that is orthogonal to the first direction;
wherein one end of the orthosis attachment system attaches to the load distribution device at a first location that is at least one of horizontally and vertically offset from a second location that attaches to a second end of the orthosis attachment system; and
wherein the offset attachment locations on the load distribution device impart one or more moments of rotation to the load distribution device.

17) The load distribution device of claim 1, wherein the wearer is a human or an animal.
